(19) United States
(12) Patent Application Publication
(10) Pub. No.: US 2006/0030621 A1
(43) Pub. Date: Feb. 9, 2006

(54) Preparation for external use on skin

(76) Inventors: Satoshi Inaoka, Osaka City (JP); Shuichi Tsunetsugu, Kobe City (JP)

Correspondence Address: CHOATE, HALL & STEWART LLP, TWO INTERNATIONAL PLACE, BOSTON, MA, 02110, US

(21) Appl. No.: 11/138,860

(22) Filed: May 26, 2005

(30) Foreign Application Priority Data
May 20, 2005 (CA) 2,508,093
May 28, 2004 (JP) JP 2004-160139

(51) Int. Cl. A61K 31/375; A61K 31/198

(52) U.S. Cl. 514/474; 514/554

(57) ABSTRACT

The present invention has as an object to provide a preparation for external use on skin in which the percutaneous absorption amount of ascorbic acid or an ascorbic acid derivative is increased and an effect on improving skin pigmentation and dullness is enhanced. In a preparation for external use on skin, comprising one or more compounds selected from ascorbic acid and ascorbic acid derivatives, by employing diglycerol and a low molecular weight betaine together, a preparation for external use on skin in which the percutaneous absorption of the ascorbic acid or the ascorbic acid derivative is increased, an effect on improving skin pigmentation and dullness, etc. is sufficiently exhibited and an excellent humectant effect is exhibited can be provided.

### BACKGROUND OF THE INVENTION

1. Field of the Invention

The present invention relates to a preparation for external use on skin which is excellent in percutaneous absorption of ascorbic acid or an ascorbic acid derivative, and has a high humectant effect.

2. Description of Related Art

It is known that ascorbic acid or an ascorbic acid derivative exhibits various effects such as anti-inflammatory effects, effects of ameliorating acne, whitening effects, anti-ageing effects, antioxidation effects, effects of stimulating cells due to acceleration of syntheses for biological components such as collagen, effects of controlling DNA damage or cell disorders of epidermal kerationocytes due to UV, and is widely employed in a preparation for external use on skin in anticipation of these effects. In order to sufficiently exhibit these effects, a method for increasing the percutaneous absorption amount of ascorbic acid or an ascorbic acid derivative and for allowing it to be effectively absorbed was needed.

A low molecular weight betaine represented by trimethylglycine is widely employed as a humectant in a preparation for external use on skin. It is known that the low molecular weight betaine enhances percutaneous absorption of a whitening component such as an ascorbic acid derivative. It is also known that, in a preparation for external use on skin containing a whitening agent, a low molecular weight betaine, and an oil component selected from silicone oils and plant oils, percutaneous absorption of the whitening agent is improved thereby enhancing an effect on improving skin pigmentation and dullness by the whitening agent, enhancing an effect on improving rough skin and humectant function and providing a good sensation in use as well as enhancing a whitening effect (Patent document 1: JP-A-2001-89321).

Diglycerol is a component employed in a preparation for external use on skin as a humectant component. It is known that by employing trimethylglycine and diglycerol together, a humectant effect is sustained (Patent document 2: JP-A-8-20520).

### SUMMARY OF THE INVENTION

The present invention has as an object to provide a preparation for external use on skin comprising ascorbic acid or an ascorbic acid derivative, in which the percutaneous absorption amount of the ascorbic acid or the ascorbic acid derivative is increased, an effect on improving skin pigmentation and dullness is sufficiently exhibited, and an excellent humectant effect is exhibited.

As a result of diligent research in order to overcome the problems described above, the present inventors discovered that, by employing diglycerol and a low molecular weight betaine together in a preparation for external use on skin comprising one or more compounds selected from ascorbic acid and ascorbic acid derivatives, a preparation for external use on skin in which the percutaneous absorption of the ascorbic acid or the ascorbic acid derivative is increased, an effect on improving skin pigmentation and dullness is sufficiently exhibited, and an excellent humectant effect is exhibited is provided.

That is, the present invention relates to preparations for external use on skin described in (1) to (3) shown in the following. (1) A preparation for external use on skin, comprising (A) one or more compounds selected from ascorbic acid and ascorbic acid derivatives; (B) diglycerol; and (C) a low molecular weight betaine. (2) The preparation for external use on skin described in (1), wherein the low molecular weight betaine is trimethylglycine. (3) The preparation for external use on skin described in (1) or (2), further comprising at least one component selected from the group consisting of a whitening component, an anti-inflammatory component, an antibacterial component, a cell stimulating component, an astringent component, an antioxidant component, an anti-ageing component, and a humectant component.

### DETAILED DESCRIPTION OF THE INVENTION

In the specification of the present application, “%” means “% by weight” unless otherwise indicated.

In the present invention, by employing one or more compounds selected from ascorbic acid and ascorbic acid derivatives together with diglycerol and a low molecular weight betaine, the percutaneous absorption amount of the ascorbic acid or the ascorbic acid derivative can be dramatically increased. For this reason, a preparation for external use on skin in which an effect of the ascorbic acid or the ascorbic acid derivative is sufficiently exhibited, and an excellent effect on improving skin pigmentation and dullness is exhibited can be provided. In addition, by using the preparation for external use on skin of the present invention, the skin can be protected from dryness, which is a unique property derived from ascorbic acid or an ascorbic acid derivative, and the moisture retention ability of the skin can be maintained and enhanced. Accordingly, the moisture retention ability of the skin is improved, skin pigmentation or dullness is improved, and skin firmness and elasticity is dramatically improved.

As the ascorbic acid or its derivative employed in the present invention, products which are commercially available as components of a preparation for external use on skin in the field of medicines, quasi drugs, or cosmetics, can be employed. In addition, the ascorbic acid or its derivative employed in the present invention is not particularly limited as long as it is employed as a component of a preparation for external use on skin in the field of medicines, quasi drugs, or cosmetics, and the compounds described above can be freely employed alone or in combination of two or more kinds thereof.

Among the ascorbic acid and the ascorbic acid derivatives of the present invention described above, ascorbic acid, ascorbyl phosphoric ester derivatives, ascorbyl sulfuric ester derivatives, ascorbyl palmitic ester derivatives, ascorbyl ether derivatives are preferable. More specific examples include ascorbyl monophosphoric esters, ascorbyl diphosphoric esters, ascorbyl triphosphoric esters, ascorbyl-2-monosulfuric ester, ascorbyl-2-disulfuric ester, ascorbyl-2-trisulfuric ester, ascorbyl monopalmitic esters, ascorbyl dipalmitic esters, ascorbyl tripalmitic esters, ascorbyl-2-glucoside, and salts thereof. In view of high safety with respect to the skin or mucosa and high effects, ascorbic acid, ascorbyl monophosphoric esters and salts thereof, ascorbyl palmitate and ascorbyl-2-glucoside, are particularly preferable.

In the present invention, any of D-, L- and DL-ascorbic acids can be employed. In the present invention, since percutaneous absorption of ascorbic acid or its derivative is dramatically improved, a water-soluble ascorbic acid or a water-soluble ascorbic acid derivative (for example, ester derivatives or ether derivatives of ascorbic acid and the like), which is generally considered difficult to be absorbed into the skin can be also preferably employed. Specific examples of the water-soluble ascorbic acid derivative include, as ester derivatives, L-ascorbyl phosphoric ester derivatives such as L-ascorbyl monophosphoric esters, L-ascorbyl diphosphoric esters and L-ascorbyl triphosphoric esters; L-ascorbyl sulfuric esters such as L-ascorbyl-2-sulfuric ester; and the like, and as ether derivatives, L-ascorbyl-2-glucoside and the like. Among these, L-ascorbic acid, L-ascorbyl phosphoric ester derivatives, L-ascorbyl-2-sulfuric ester, and L-ascorbyl-2-glucoside are preferable. In view of high safety with respect to the skin or mucosa and high effects, L-ascorbic acid, L-ascorbyl monophosphoric esters, and L-ascorbyl-2-glucoside, are particularly preferable.

In addition, the ascorbic acid or its derivative may be employed as a pharmaceutically acceptable salt. As examples thereof, mention may be made of, for example, salts with an organic base (for example, salts with a tertiary amine such as a trimethylamine salt, a triethylamine salt, a monoethanolamine salt, a triethanolamine salt and pyridine salt, basic ammonium salts such as arginine, and the like), salts with an inorganic base (for example, ammonium salts, alkali metal salts such as a sodium salt and a potassium salt, alkaline earth metal salts such as a calcium salt and a magnesium salt, an aluminum salt, and the like). In particular, preferable salts are a sodium salt, and a potassium salt. As specific examples thereof, mention may be made of sodium ascorbate, sodium ascorbyl monophosphate, sodium ascorbyl diphosphate, sodium ascorbyl triphosphate, sodium ascorbyl-2-sulfate and the like.

In the preparation for external use on skin of the present invention, the blending amount of the ascorbic acid or its derivative can preferably range from 0.1 to 30% by weight with respect to the total weight of the preparation for external use on skin. Within the range described above, the amount can be appropriately selected, depending on the various desired effects of the ascorbic acid or its derivative and depending on use of the preparation for external use on skin. In view of the effects of the present invention, the amount is more preferably in the range of from 3 to 25% by weight, and is particularly preferably in the range of from 5 to 20% by weight.

The diglycerol employed in the preparation for external use on skin of the present invention is a known compound, and the blending amount of the diglycerol employed in the preparation for external use on skin is not particularly limited as long as the effects of the present invention can be exhibited, and can be appropriately selected as long as the effects of the present invention can be exhibited. The amount may commonly range from 1 to 95% by weight with respect to the total weight of the preparation for external use on skin, may preferably range from 1 to 50% by weight, may more preferably range from 1 to 20% by weight, and may particularly preferably range from 1 to 10% by weight.

The low molecular weight betaine employed in the preparation for external use on skin of the present invention means one having a molecular weight of 200 or less, and forming an amphoteric ion in the molecule. Specific examples thereof include a quaternary ammonium base, a quaternary phosphonium base, a tertiary sulfonium base, and the like. They exhibit little surfactant activity. Among these, an N,N,N-trialkylamino acid represented by formula (1) shown below is preferable.

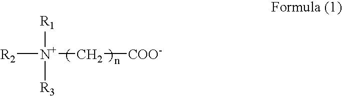

wherein R₁, R₂, and R₃ independently represent an alkyl group having 1 to 6 carbon atoms; and n represents 1 to 6.

As R₁ to R₃, a straight or branched chain alkyl group having 1 to 6 carbon atoms can be widely employed. That is, as examples thereof, mention may be made of, a methyl group, an ethyl group, a propyl group, an isopropyl group, a butyl group, an isobutyl group a sec-butyl group, tert-butyl group, a pentyl group, an isopentyl group, a neopentyl group, a tert-pentyl group, a hexyl group, an isohexyl group, a 3-methylpentyl group, 2,2-dimethylbutyl group, a 2,3-dimethylbutyl group, and the like. R₁ to R₃ may be the same or different.

In particular, in the case of n=1, examples thereof include trimethylglycine, triethylglycine, tripropylglycine, and triisopropylglycine; in the case of n=2, examples thereof include trimethyl-beta-alanine; and in the case of n=3, examples thereof include trimethyl-gamma-aminobutyric acid, and the like. Trimethylglycine is preferable.

In addition, the low molecular weight betaines described above may have substituents. In particular, in the case of n=1, as examples thereof, mention may be made of N,N,N-trimethylalanine, N,N,N-triethylalanine, N,N,N-triisopropylalanine, N,N,N-trimethylmethylalanine, carnitine, acetyl carnitine, and the like. Carnitine is preferable.

In the preparation for external use on skin of the present invention, the blending amount of the low molecular weight betaine preferably ranges from 0.5 to 10% by weight with respect to the total weight of the preparation for external use on skin. Within the range described above, the amount can be appropriately selected depending on various desired effects of ascorbic acid or depending on use of the preparations for external use on skin. In view of the effects of the present invention, the amount is preferably in the range of from 0.5 to 9% by weight, and is particularly preferably in the range of from 1 to 8% by weight. If the amount is below 0.1% by weight, the effects cannot be exhibited in some cases. On the other hand, if the amount exceeds 10% by weight, a poor sensation in use may be provided in some cases.

In the preparation for external use on skin of the present invention, in addition to the ascorbic acid or its derivative described above, various components such as a whitening component, an anti-inflammatory component, an antibacterial component, a cell stimulating component, an astringent component, an antioxidant component, a component for ameliorating acne, an anti-ageing component, a component for accelerating syntheses for biological ingredients such as collagen, a blood circulation accelerator component and a humectant component can be blended alone or in combination of two or more kinds thereof in order to further add other useful effects to the preparation for external use on skin. Preferably, components are one or more kinds of the whitening component, the anti-inflammatory component, the antibacterial component, the cell stimulating component, the astringent component, the antioxidant component, the anti-ageing component, and the humectant component. The components described above are not particularly limited as long as they are conventionally employed or will be employed in the future as the components of preparations for external use on skin in the field of medicines, quasi drugs, or cosmetics. As the components described above, any components can be appropriately selected and be employed.

For example, as examples of whitening components, mention may be made of arbutin; ellagic acid; phytic acid; rucinol; chamomile ET; vitamins such as vitamin A or derivatives thereof, vitamin E or derivatives thereof, pantothenic acid or derivatives thereof, and the like; and the like. Among these, as preferable examples thereof, mention may be made of pantothenic acid or derivatives thereof, ellagic acid, phytic acid, vitamin A or derivatives thereof, and vitamin E or derivatives thereof. The whitening components described above can be employed alone or in combination of two or more kinds thereof.

Plant components exhibiting whitening effects may be employed as whitening components. As examples of the plant components described above, mention may be made of components derived from plants such as iris, almond, aloe, gingko, oolong tea, rose hips, Scutellaria baicalensis, Coptis japonica, Hypericum erectum, dead nettle, seaweed, Pueraria lobata, cape jasmine, Sophora flavescens, chlorella, Schlechtendaria chinensis, wheat, rice, rice germ, oryzanol, rice bran, Asarum sieboldii, Zanthoxyli fructus, perilla, Paeoniae radix, Cnidium officinale, Morus australis, soybean, fermented soybean, tea, Angelica sinensis, Calendula officinalis, garlic, Hamamelis virginiana, safflower, Paeonia suffruticosa, Coix lacryma-jobi, Angelica sinensis [sic], Salvia leucantha, Uncaria gambir, asebiwarabi [phonetic spelling], Podocarpus macrophyllus, Flammulina velutipes, Diospyros kaki, Catalpa ovata, black bean, Gentiana amarella, Scrophularia buergeriana, Smilax medoca, snap bean, shokuma [phonetic spelling], Paris polyphylla, sage, Peuceadanum praeruptorum, Japanese radish, Ericaceae, Lespedeza homoloba, toshin [phonetic spelling], Picrasma quassioides, parsley, holly, hop, Lespedeza cyrtobotrya, clove, Glycyrrhiza glabra, and the like. Preferable are plant components derived from iris, aloe, gingko, oolong tea, rose hips, Scutellaria baicalensis, Coptis japonica, Hypericum erectum, dead nettle, seaweed, Pueraria lobata, cape jasmine, Sophora flavescens, Schlechtendaria chinensis, wheat, rice, rice bran, Asarum sieboldii, Zanthoxyli fructus, perilla, Paeoniae radix, Cnidium officinale, Morus australis, tea, Angelica sinensis, Calendula officinalis, Hamamelis virginiana, safflower, Paeonia suffruticosa, Coix lacryma-jobi, Salvia leucantha, Uncaria gambir, Flammulina velutipes, Diospyros kaki, Catalpa ovata, black bean, Gentiana amarella, Smilax medoca, snap bean, Paris polyphylla, sage, Peuceadanum praeruptorum, Japanese radish, Ericaceae, Lespedeza homoloba, toshin [phonetic spelling], Picrasma quassioides, parsley, holly, hop, clove, Glycyrrhiza glabra, and Angelica sinensis [sic]. More preferable are plant components derived from iris, aloe, gingko, rose hips, Scutellaria baicalensis, Coptis japonica, Hypericum erectum, cape jasmine, Sophora flavescens, rice, rice bran, Asarum sieboldii, Paeoniae radix, Cnidium officinale, Morus australis, tea, Angelica sinensis, Calendula officinalis, Hamamelis virginiana, safflower, Paeonia suffruticosa, Salvia leucantha, Uncaria gambir, Flammulina velutipes, Diospyros kaki, sage, Japanese radish, Ericaceae, parsley, hop, Glycyrrhiza glabra, and Coix lacryma-jobi. In the case of employing the plant components described above in the preparation for external use on skin of the present invention, the form of the plant components is not particularly limited. In general, the form such as a plant extract, an essential oil, or the like, can be employed.

As examples of anti-inflammatory components, mention may be made of allantoin, calamine, glycyrrhizic acid or derivatives thereof, glycyrrhetic acid or derivatives thereof, zinc oxide, guaiazulene, tocopherol acetate, pyridoxine hydrochloride, menthol, camphor, turpentine oil, indomethacin, salicylic acid or derivatives thereof, and the like. Preferable are allantoin, glycyrrhizic acid or derivatives thereof, glycyrrhetic acid or derivatives thereof, guaiazulene, and menthol.

As examples of antibacterial components, mention may be made of chlorhexidine, salicylic acid, benzalkonium chloride, acrinol, ethanol, benzethonium chloride, cresol, gluconic acid and derivatives thereof, povidone iodine, potassium iodide, iodine, isopropyl methylphenol, triclocarban, triclosan, sensitizing dye No. 101, sensitizing dye 201, paraben, phenoxyethanol, 1,2-pentane diol, alkyldiaminoglycine hydrochloride, and the like. As preferable examples thereof, mention may be made of benzalkonium chloride, benzethonium chloride, gluconic acid and derivatives thereof, isopropyl methylphenol, triclocarban, triclosan, sensitizing dye No. 101, sensitizing dye No. 201, paraben, phenoxyethanol, 1,2-pentane diol, alkyldiaminoglycine hydrochloride, and the like. More preferable are benzalkonium chloride, gluconic acid and derivatives thereof, benzethonium chloride, and isopropyl methylphenol.

As examples of cell stimulating components, mention may be made of amino acids such as γ-aminobutyric acid, ε-aminopuronic acid, and the like; vitamins such as retinol, thiamine, riboflavin, pyridoxine hydrochloride, pantothenic acid, and the like; alpha-hydroxylic acids such as glycolic acid, lactic acid, and the like; tannin, flavonoid, saponin, allatoin, sensitizing dye No. 301, and the like. Preferable are amino acids such as γ-aminobutyric acid, ε-aminopuronic acid, and the like; and vitamins such as retinol, thiamine, riboflavin, pyridoxine hydrochloride, pantothenic acid, and the like.

As examples of astringent components, mention may be made of metal salts such as alum, chlorohydroxyaluminum, aluminum chloride, allantoin aluminum salt, zinc sulfate, aluminum potassium sulfate, and the like; and organic acids such as tannic acid, citric acid, lactic acid, succinic acid, and the like. Preferable are alum, chlorohydroxyaluminum, aluminum chloride, allantoin aluminum salt, aluminum potassium sulfate, and tannic acid.

As examples of antioxidant components, mention may be made of tocopherol and derivatives thereof, butylhydroxyanisole, dibutylhydroxytoluene, sodium hydrogen sulfite, erythorbic acid and salts thereof, flavonoid, glutathione, glutathione peroxidase, glutathione-S-transferase, catalase, superoxide dismutase, thioredoxin, taurine, thiotaurine, hypotaurine, and the like. Preferable are tocopherol and derivatives thereof, thiotaurine, hypotaurine, thioredoxin, and flavonoid.

As examples of anti-ageing components, mention may be made of retinoid (retinol, retinoic acid, retinal, and the like), pangamic acid, kinetin, ursolic acid, an extract of Curcuma longa, sphingosine derivatives, silicon, silicic acid, N-methyl-L-serine, mevalonolactone, and the like. Preferable are retinoid (retinol, retinoic acid, retinal, and the like), and kinetin.

As examples of humectant components, mention may be made of amino acids and derivatives thereof such as alanine, serine, leucine, isoleucine, threonine, glycine, proline, hydroxyproline, glucosamine, theanine, and the like; peptides such as collagen, collagen peptide, gelatin, and the like; polyhydric alcohols such as glycerol, 1,3-butylene glycol, propylene glycol, polyethylene glycol, and the like; sugar alcohols such as sorbitol and the like; phospholipids such as lecithin, hydrogenated lecithin, and the like; mucopolysaccharides such as hyaluronic acid, heparin, chondroitin, and the like; components based on NMF such as lactic acid, sodium pyrrolidone carbonate, urea, and the like; polyglutamic acid, and the like. Preferable are alanine, serine, glycine, proline, hydroxyproline, glucosamine, theanine, collagen, collagen peptide, glycerol, 1,3-butylene glycol, hydrogenated lecithin, hyaluronic acid, heparin, chondroitin, lactic acid, sodium pyrrolidone carbonate, and polyglutamic acid.

In the case of employing humectant components, the ratio thereof blended in the preparation for external use on skin of the present invention commonly ranges from 0.1 to 10% by weight, preferably ranges from 0.5 to 5% by weight, and more preferably ranges from 0.5 to 5% by weight [sic].

In the preparation for external use on skin of the present invention, in addition to the components described above, surfactants, solubilizing components, fats and oils, sugars, or percutaneous absorption accelerator components can be further blended. In particular, by blending surfactants, solubilizing components, or fats and oils, stability of the water-soluble ascorbic acid in an aqueous medium, efficacy thereof, and sensation in use can be improved.

As examples of surfactants employed herein, mention may be made of various nonionic surfactants, examples of which include polyoxyethylene (hereinafter, referred to as POE) branched alkyl ethers such as POE octyldodecyl alcohol, POE 2-decyltetradecyl alcohol, and the like; POE alkyl ethers such as POE oleyl alcohol ether, POE cetyl alcohol ether, and the like; sorbitan esters such as sorbitan monooleate, sorbitan monoisostearate, sorbitan monolaurate, and the like; POE sorbitan esters such as POE sorbitan monooleate, POE sorbitan monoisostearate, POE sorbitan monolaurate, and the like; glycerol fatty acid esters such as glycerol monooleate, glycerol monostearate, glycerol monomyristate, and the like; POE glycerol fatty acid esters such as POE glycerol monooleate, POE glycerol monostearate, POE glycerol monomyristate, and the like; POE hardened castor oil fatty acid esters such as POE dihydrocholesterol ester, POE hardened castor oil, POE hardened castor oil isostearate, and the like; POE alkyl aryl ethers such as POE octyl phenyl ether, and the like; glycerol alkyl ethers such as monoisostearyl glyceryl ether, monomyristyl glyceryl ether, and the like; POE glycerol alkyl ethers such as POE monostearyl glyceryl ether, POE monomyristyl glyceryl ether, and the like; polyglycerol fatty acid esters such as diglyceryl monostearate, decaglyceryl decastearate, decaglyceryl decaisostearate, diglyceryl diisostearate, and the like; or natural surfactants such as lecithin, hydrogenated lecithin, saponin, surfactin sodium salt, cholesterol, bile acid, and the like; and the like. The surfactants described above can be employed alone or in combination of two or more kinds thereof.

As fats and oils, they are not particularly limited as long as they are those employed as components of preparations for external use in the field of medicines, quasi drugs, or cosmetics. As examples thereof, mention may be made of synthetic oils such as middle-chain fatty acid triglyceride, and the like; vegetable oils such as soybean oil, rice oil, rapeseed oil, cotton seed oil, sesame oil, safflower oil, castor oil, olive oil, cacao butter, camellia oil, sunflower oil, palm oil, linseed oil, perilla oil, shea oil, saru [phonetic spelling] oil, coconut oil, Japan wax, jojoba oil, grape seed oil, avocado oil, and the like; animal oils such as mink oil, yolk oil, beef tallow, milk fat, lard, and the like; waxes such as beeswax, spermaceti wax, lanolin, carnauba wax, candelilla wax, and the like; hydrocarbons such as liquid paraffin, squalene, squalane, microcrystalline wax, ceresin wax, paraffin wax, vaseline, and the like; natural or synthetic fatty acids such as lauric acid, myristic acid, stearic acid, oleic acid, isostearic acid, behenic acid, and the like; natural or synthetic higher alcohols such as cetanol, stearyl alcohol, hexyldecanol, octyldecanol, lauryl alcohol, and the like; esters or ethers such as isopropyl myristate, isopropyl palmitate, octyldodecyl myristate, octyldodecyl oleate, cholesterol oleate, and the like; silicone oils; and the like. The fats and oils described above can be employed alone or in combination of two or more kinds thereof.

As sugars, they are not particularly limited as long as they are those employed as components of preparations for external use in the field of medicines, quasi drugs, or cosmetics. As examples thereof, mention may be made of monosaccharides (such as glucose, galactose, mannose, ribose, arabinose, xylose, deoxyribose, fructose, ribulose, lyxose, and the like), disaccharides (such as sucrose, trehalose, lactose, maltose, cellobiose, and the like), oligosaccharides (such as lactulose, raffinose, pullulan, and the like), cellulose and derivatives thereof (such as methylcellulose, ethylcellulose, hydroxyethylcellulose, hydroxypropylcellulose, carboxymethylcellulose, carboxyethylcellulose, nitrocellulose, and the like), polymer sugars (such as chondroitin sulfate, hyaluronic acid, dermatan, heparan, heparin, keratan, and salts thereof (pharmaceutically or physiologically acceptable salts such as sodium chondroitin sulfate, sodium hyaluronate, dermatan sulfate, haparan sulfate, keratan sulfate, and the like), and the like), and sugar alcohols (such as mannitol, xylitol, erythritol, pentaerythritol, maltitol, sorbitol, polydextrose, and the like), and in addition, xylose, inositol, dextrin and derivatives thereof, honey, a muscovado extract, and the like. The sugars described above may be employed alone or in combination of two or more kinds thereof.

In the preparation for external use on skin of the present invention, various components which are generally employed as components of preparations for external use in the field of medicines, quasi drugs, or cosmetics, such as amino acids, reducers for irritation, thickening agents, preservatives, UV controlling agents, coloring agents, pH adjustors, perfumes, and the like, can be blended within a quantitative and qualitative range which does not impair the quality such as apparent stability, viscosity, and the like, and does not impair the effects of the present invention. The components described above can be freely employed alone or in combination of two or more kinds thereof.

The preparation for external use on skin of the present invention can be prepared in a preferable form of a paste, a mousse, a gel, a liquid, a milky lotion, a cream, a sheet (base material carrier), an aerosol, a spray, or the like, by blending and mixing each of optional components described above if necessary, and in addition, blending another solvent or a base agent of a preparation for external use generally employed or the like if necessary. They can be produced in a conventional method known in the art.

The preparation for external use on skin of the present invention may commonly have liquid properties at pH 1 to pH 8. In view of stability of the water-soluble ascorbic acid, low irritation with respect to the skin and mucosa, and a good sensation in use on the skin, it is in the range of preferably from pH 2 to pH 7, and more preferably from pH 2 to pH 6.

The preparation for external use on skin of the present invention can be formed into various compositions for external use in the field of cosmetics, medicines for external use, or quasi drugs for external use, including makeup cosmetics such as foundations, lipsticks, mascaras, eye shadows, eyeliners, eyebrow colors and nail varnishes; base cosmetics such as milky lotions, creams, lotions, oils and facial packs; cleansing compositions such as face cleansing compositions, cleansers and body washes; underarm deodorants, athlete's foot remedies, anti-itching preparations, wound healing preparations, dry bathing preparations, cleaning preparations, anti-inflammatory analgesic preparations, acne remedies, hemorrhoidal preparations, sterilizing preparations, whitening preparations, UV controlling preparations, and the like. In view of enhancing effects on the skin, the preparation of the present invention is preferably employed as a product for applying on the outer skin, such as a percutaneous preparation.

### EXAMPLES

In the following, the present invention is described in detail based on Examples and Test Examples. It should be understood that the present invention is not limited to the Examples and the like. In each of the composition examples described below, “%” means % by weight (W/W), unless otherwise indicated.

### Test Example 1

The dorsal skin of a hairless mouse was pinched and fixed between percutaneous absorption cells, and the cell at the receptor side was filled with saline and the cell at the donor side was filled with each of the preparations shown in Table 1. The solutions in both cells were incubated at 37° C. while being agitated with a stirrer bar. After 24 hours, the concentration of ascorbic acid in the solution at the receptor side was measured by high performance liquid chromatography. The measurement results are shown in Table 1. The results are represented by the relative ratio based on the permeation amount of ascorbic acid in Comparative Example 1 (solution containing only ascorbic acid), which was defined as 1.

TABLE 1

| | Comparative | Comparative | Comparative | | |
|---|---|---|---|---|---|
| | Example 1 | Example 2 | Example 3 | Example 1 | Example 2 |
| L-ascorbic acid | 10% | 10% | 10% | 10% | 10% |
| Diglycerol | — | 5% | — | 5% | 10% |
| Trimethylglycine | — | — | 5% | 5% | 2% |
| Water | 90% | 85% | 85% | 80% | 78% |
| Relative ratio of percutaneous | 1.0 | 1.2 | 1.5 | 2.0 | 1.8 |
| permeation amount | | | | | |

From the results of the test, it could be confirmed that, in the case of combining diglycerol and trimethylglycine, the permeation amount of ascorbic acid, namely, percutaneous absorption amount thereof was increased. The preparation for external use on skin comprising ascorbic acid together with diglycerol and trimethylglycine can deliver ascorbic acid into the skin more; thus, an effect that ascorbic acid possesses can be sufficiently exhibited.

### Test Example 2

Ten panelists were asked to actually use the preparations of Comparative Example 1 and Example 2, and to perform sensory evaluation in terms of improvement of the skin pigmentation, improvement of the skin dullness, an elasticity sensation of the skin, and a moisturizing sensation of the skin. They were asked to apply the preparations to the entire face twice a day after washing the face and treating the skin with the lotion usually employed. The evaluation was performed by first, employing the preparation of Comparative Example 1 continuously for 2 weeks, then, employing the preparation of Example 2 continuously for 2 weeks. As the evaluation criteria, the panelists were asked to evaluate whether the preparation of Example 2 is better than, almost equal to, or inferior to that of Comparative Example 1, and the cases where the ratio of the panelists who answered that the preparation of Example 2 is better than that of Comparative Example 1 is 80% or higher was defined as ◯◯, from 60 to 80% was defined as ◯, from 50 to 60% was defined as Δ, and lower than 50% was defined as x. In addition, the same sensory evaluation was performed by asking panelists to actually use the preparations of Comparative Example 1 and Comparative Example 3. The results are shown in Table 2.

TABLE 2

| | Comparative | |
|---|---|---|
| | Example 3 | Example 2 |
| Improvement of the skin pigmentation | Δ | ◯ |
| Improvement of the skin dullness | ◯ | ◯◯ |
| Elasticity sensation of the skin | ◯ | ◯◯ |
| Moisturizing sensation of the skin | Δ | ◯◯ |

From the results of the test, it was confirmed that, in the case of the preparation of Example 2 in which diglycerol and trimethylglycine were contained together with ascorbic acid, the percutaneous absorption of ascorbic acid was increased, an effect that ascorbic acid possesses was sufficiently exhibited, whereby the skin dullness and pigmentation was improved, elasticity of the skin was increased, a moisturizing sensation of the skin was improved, and an excellent effect on restoring the skin's moisture and the firm appearance of the skin was exhibited.

What is claimed is:

1. A preparation for external use on skin, comprising:
(A) one or more compounds selected from ascorbic acid and ascorbic acid derivatives;
(B) diglycerol; and
(C) a low molecular weight betaine.

2. The preparation for external use on skin according to claim 1, wherein the low molecular weight betaine is trimethylglycine.
